(19) United States
(12) Patent Application Publication
(10) Pub. No.: US 2007/0286804 A1
(43) Pub. Date: Dec. 13, 2007

(54) Method, materials and apparatus for investigating asthma using dust mite allergen

(75) Inventors: Piyush Ranchodbhai Patel, Mississauga (CA); Anne Marie Salapatek, Mississauga (CA)

Correspondence Address: BENESCH, FRIEDLANDER, COPLAN & ARONOFF LLP;ATTN: IP DEPARTMENT DOCKET CLERK, 2300 BP TOWER, 200 PUBLIC SQUARE, CLEVELAND, OH, 44114, US

(73) Assignee: Allied Research International Inc.

(21) Appl. No.: 11/450,786

(22) Filed: Jun. 9, 2006

(51) Int. Cl. A61K 49/00

(52) U.S. Cl. 424/9.1

(57) ABSTRACT

The present invention comprises a method, materials and apparatus for investigating asthma in humans using dust mite allergen. The dust mite allergen is prepared to be of a controlled size such that particles are respirable, effectively aerosolized, and deliver a significant amount of allergen. The dust mite allergen is applied in a controlled manner within an environmental exposure chamber to elucidate the etiological links between dust mite allergen concentration and asthma response in humans. The environmental exposure chamber is specially-designed to promote homogeneity of allergen concentration. Preferably, the dust mite allergen preparation comprises particles having an average diameter of less than 25 microns, and more preferably 5-10 microns. Correlation between aerosolized particle count and allergen concentration enables “real-time” allergen concentration estimates without the use of expensive and time-consuming assay techniques.

### FIELD OF THE INVENTION

This invention relates to novel methods, materials and apparatus for investigating asthma in humans. More particularly, the present invention relates to novel methods, materials and apparatus for investigating asthma in humans using dust mite allergen.

### BACKGROUND OF THE INVENTION

Asthma is a chronic inflammatory disease of the airways characterized by variable airflow obstruction and airway hyper-responsiveness with clinical signs such as recurrent episodes of wheezing, breathlessness, chest tightness, and coughing. As asthma morbidity and mortality have increased, so has the importance of controlling this disease. Asthma is estimated to affect between 100 and 150 million people worldwide. In 1998 approximately 180,000 deaths worldwide were attributed to asthma. In Western Europe, the prevalence of asthma has doubled during the 10-year period preceding 1998. In the United States, asthma affects an estimated 14.6 million people (6% of the population) and the prevalence of asthma has increased by 60% since the early 1980s. In 1997 there were over 5,000 deaths in the United States attributed to asthma. Asthma is the third leading cause of preventable hospitalizations in the United States. Therefore, it is apparent that asthma is a significant disease from both a medical and economic perspective.

The inhalation of dust in the household or at the workplace has been recognized as potentially hazardous for homeowners and workers, respectively. Particularly, it has been shown that when dust is disturbed occupants are exposed transiently to higher dust borne mite allergens. It has been roundly suggested that dust mite allergen present in household dusts is the major allergen causing allergic asthma today.

Despite the epidemic rise in asthma rates world-wide, on average rising 50 percent every decade, there is no consensus on the cause. There are currently two opposing theories. One theory, the “hygiene hypothesis”, suggests that the recent rise in allergic disease among children in affluent societies is due to a lack of exposure to infections and unhygienic contact such as dust mite allergen such that children do not acquire immunity to them, thereby leading to the increased incidence of allergy and asthma.

The other allergen overexposure theory proposes that components of a Western lifestyle are increasingly placing us in static, artificial environs which includes increased time indoors and a sedentary lifestyle which lead to increased and longer duration exposures to allergens such as dust mite allergen which in turn are in part responsible for the increased incidence of allergy and asthma seen particularly in industrialized nations of Western societies.

Under either scenario, a relationship between dust mite allergen and asthma clearly exists. However, as is evident from the polarity of the causative theories, little is known about the relevant concentrations of airborne mite allergens and their clinical impact. In particular, little is known about the exact levels of dust mite allergen, which are significant in the etiology, or progression, of asthma.

It is thought that proteolytic activity is the central biochemical property that endows these molecules with intrinsic allergenicity. In particular, it is the cysteine protease region of dust mite, Der p1, that is the major allergenic molecules responsible for the increase in asthma and atopic conditions worldwide. These proteases induce Th2-driven inflammatory responses in the airways by disrupting the epithelial cell junctions so that these, and other molecules, gain access to, and alter the function of, underlying cells of the innate immune system (dendritic cells, mast cells, basophils and macrophages) and B and T cells. Another mechanism of dust mite allergen activity recently proposed is through their interactions at proteinase-activated receptors (or “PAR”), a family of G protein-coupled receptors that are widely distributed in the mammalian body. In the respiratory systems, PARs, particularly PAR-2 and PAR-1, are expressed in the epithelial and smooth muscle cells. In the lung epithelial cells, PAR-2 can be activated by exogenous proteinases including house dust mite allergens. Clinical evidence also suggests possible involvement of PARs, particularly PAR-2, in respiratory diseases. PARs thus appear to play critical roles in the respiratory systems, and respiratory diseases including asthma.

There have been two major hurdles to the understanding of dust mite allergen exposures.

First, it is evident that there is a range of exposure level and duration to dust mite allergen in the natural indoor setting. A number of studies have been conducted to measure ‘typical’ exposures to dust mite allergens in the home, daycare, school and work with comparisons made between different socio-economic groups. However, exposures to dust mite allergen can vary widely depending on whether dust is settled or disturbed or the level of dust mite allergen in bedding and in the bedroom, a site of long duration exposure. These findings indicate that the level and duration of exposure to dust mite allergen in any exposure study will be critical and will have to be tightly controlled and monitored.

Secondly, few clinical trials on exposure to dust mite allergen in controlled environments have been performed, largely due to issues of safety and possibility for adverse reaction, which in the asthmatic is a real clinical concern. Patients with asthma require special consideration due to the nature of the condition, which can be intolerably exacerbated with dust mite allergen exposure. For this reason, subjects with at least moderate asthma are usually excluded from studies. See, for example, the U.S. Food and Drug Administration guidelines. However, the European Agency for Evaluation of Medical Products, EMEA, allows inclusion of moderately asthmatic subjects for the purpose of obtaining safety data. In this case, patients with mild and intermittent asthma have been admitted to seasonal (“SAR”) and perennial allergic rhino-conjunctivitis (“PAR”) studies and can be studied under dust mite allergen exposure conditions which are tightly controlled and monitored.

These experimental hurdles have resulted in the performance of few dust mite allergen exposure studies to date due in large part to the inability to ensure subject exposure to house dust mite allergen in a continuous, controlled and long-term challenge. This has resulted from the following biophysical barriers.

Dust mite allergen, unlike pollen allergens, is found on irregularly-shaped particles of large aerodynamic diameter. This makes dust mite allergen bearing particles difficult to aerosolize as well as to maintain at constant airborne concentrations.

Further, a significant barrier to achieving stable allergen exposures has been the inability to monitor airborne dust mite allergen concentrations in real-time, since the established method of measuring dust mite allergen concentrations, Enzyme-Linked Immuno-Sorbent Assay (or “ELISA”, discussed further below), is both time-consuming and costly.

For at least these reasons, most of the few prior studies have examined the effects of dust mite allergen in patients with PAR and only one study on asthmatics.

The main method for the detection and quantification of dust mite allergen is ELISA. ELISA is a known and established method of estimating dust mite allergen in solution. The method used in these assays is called Sandwich ELISA and it is based on the principle of antibody-allergen interaction. Essentially, to utilize this assay, one antibody (the “capture” antibody) is purified and bound to a solid phase attached to the bottom of a plate well. Allergen is then added and allowed to complex with the bound antibody. Unbound products are then removed with a wash, and a labelled second antibody (the “detection” antibody) is allowed to bind to the antigen, thus completing the “sandwich”. The assay is then quantitated by measuring the amount of labelled second antibody bound to the matrix, through the use of a colorimetric substrate.

Due to the variable exposure in the home, ideally research is needed to study the effects of dust mite allergen concentration in an EEC in which airborne allergen concentration is uniformly controlled and over set exposure times.

To study the effect of dust mite allergen, spent dust mite cultures are derived from living dust mites (specifically Dermatophagoides pteronyssinus and Dermatophagoides farinae) which are cultured on a nutritive bed mounted over a grate to allow collection of the dust mite powders. These cultures contain particles of mite parts, feces, and other culture media components. An important component of the dust is dust mite allergen, which is present on the surface of dust particles. These dust mite allergens correspond to peptide sequences which have been isolated and shown experimentally to have allergenic properties. The exact allergens present are dependent on mite species cultured such that Der p1 is derived from Dermatophagoides pteronyssinus and Der f1 is derived from Dermatophagoides farinae. Particle sizes for either Der p1 or Der f1 range from submicron to over 100 microns.

With respect to particle size, it should be understood that particle penetration into the respiratory tree and lungs is dependent on particle size. Particles 2.5 microns in diameter or less are referred to as fine particulate matter, or PM2.5, and are penetrable to the terminal of the respiratory tree, the lungs. Coarse particulate matter, or PM10, is sized between 2.5 and 10 microns in diameter, and are known to lodge predominantly in the mid to lower respiratory tree. Due to evidence that there is a link between high levels of PM2.5 or PM10 and respiratory irritation and disease, the EPA has recommended that occupational daily exposures to these particles be less than 65 μg/m³ and 75 μm³, respectively.

Under household conditions, a dust particle on which dust mite allergen is present is seen generally in particles 10 microns or greater, whereas for other household allergens, such as cat allergen, these are carried on particles less than 5 microns.

As mentioned, the etiology of asthma involves the narrowing of the respiratory tract such that insufficient air passes to the lungs resulting in reduced oxygenation and increased carbon dioxide retention which leads to some of the most devastating symptoms of asthma, such as inability to overcome the increased respiratory resistance and inability to appropriately aerate the lungs and breathe. Therefore, particles bearing allergen which are sized in the 5 to 10 micron range are likely the key determinants of asthma development and attacks. These particles can penetrate and lodge in the lowest part of the respiratory tree (not appreciably in the lungs) resulting in airway inflammatory response and narrowing of the airway, the latter due to constriction of the underlying smooth muscle in response to allergen application.

With respect to the disease itself, the two fundamental features of asthma are variability of airway caliber and increased bronchial hyperreactivity to non-specific stimuli (or “BHR”). The degree of BHR has been related to the number of inflammatory cells in the blood and airways, suggesting that the physiological dysfunctions in asthma are caused by an underlying inflammatory process. Allergens are considered to be important inducers of BHR in allergic patients.

Investigation of the disease via the introduction of allergen to an allergenic patient can enable the determination of the efficacy of treatment options. In particular, following high dose allergen exposure in an allergic patient, an early asthmatic reaction (or “EAR”) occurs immediately after exposure and after 3-7 hours, a late asthmatic reaction (or “LAR”) may develop in some patients. The EAR is transient and resolves in one hour. The LAR, on the other hand, is associated with influx of inflammatory cells into the blood and airways as well as the increase of BHR, which sometimes can persist for several days.

However, as mentioned, hitherto such clinical investigative models have been relatively limited.

A high dose allergen provocation model has been used to study pathophysiological mechanisms of allergic reactions and to evaluate the effects of novel therapeutic agents being developed for asthma, but this model has been criticized for being too experimental.

Repeated low dose allergen exposure with a nasal challenge model via a nebulizer has also been used with house dust mite exposure, this more closely resembles exposure to dust mite in the natural setting with intermittent exposures to moderate doses while dusting or making the bed. This can be done in mild asthmatics where the amount of allergen administered daily is about 25% of the dose of allergen that has caused an EAR and LAR. This dose does not lead to significant asthma symptoms, but the BHR is increased. However, this model fails to provide dust mite exposures in a more natural setting like that found in typical indoor environs.

Investigations can be carried out employing environmental exposure chambers, or “EECs”, which are chambers designed for the study of aerosol particulates. In the past, EECs have been used to look at industrial type exposures to particulate pollutants. For example, aerosolization studies were done with small particulate pollutants in chambers designed by Bryan & Blackmore, which were subsequently used by Briscoe & Day used as a model for the first designs of allergen chambers.

The first allergen really studied in this manner was ragweed pollen, which has been initially described and reviewed by Day et al. in 1999. In this study, Day describes a design of a chamber in which ragweed pollen is aerosolized and subjects with a history of allergy to ragweed are exposed in a controlled manner to this allergen.

Up until now, most studies have been conducted to study the effects of drug therapies on the symptoms of allergic rhinitis, particularly SAR.

Krug et al. carried out the most extensive published validation of a pollen exposure chamber. In this study, a laser counter was used to count pollen particles, but no relationship between particle size or counts was made to allergen content.

Other methods have been used to expose subjects in a controlled fashion to allergens, such as nasal allergen challenge (nasal lavage) and nebulization of allergen via an inhaler.

However, the use of chambers to study exposure to other non-pollen allergens such as dust mite allergen has been limited not only in the number of studies published, but also there have been few details provided about the exact methods or chamber designs for non-pollen allergen chambers. Most recently a review was published by Day et al. which provided some insight into the use of environmental chambers for investigating allergenic response, but that review provides little detail with respect to the design and use of the chamber for dust mite allergen challenge.

Non-pollen allergens have included mostly cat allergen or dust mite allergen studies. Most recently, Berkowitz et al. exposed patients to cat allergen to produce cat allergen induced rhinitis and study the effects of the antihistamine, fexofenadine on subjects with PAR. In this study and its predecessors a live cat challenge model is used for exposing patients to the cat allergen. The methods proved imprecise resulting in a wide range of individual cat allergen exposures, from 94.0 to as high as 9,101.0 ng/m³ Fel d 1, leading to an inability to comment rigorously on the effectiveness of the test drug treatment in the individual. This indicates the importance of tightly controlling the airborne allergen levels.

With respect to dust mite, dust mite allergen exposure has been tested in a few published articles in which typical allergic symptom outcomes were measured in patients with PAR not asthma.

A first study, conducted by Horak et al. in 1994, exposed 12 patients to 40 ng Der p1 per cubic meter. The concentration of dust particles was monitored at 5 minute intervals by counting particles to a level of 4000 particles/m³. The preceding abstract to this paper stated that the estimated amount of allergen per particle, Der p1, was 0.02 ng. In neither abstract or publication was it stated what detection methods were used to measure or maintain this particle count, nor was particle count or particle size data provided over the course of the 4 hour study which would indicate the range of particle exposures.

High exposures to respirable particles sized less than 10 microns, PM10, or less than 2.5 microns, PM2.5 have been linked with respiratory disease and for these reasons the U.S. Environmental Protection Agency (EPA) has set out guidelines for daily exposures to PM10 and PM2.5 (discussed below). Therefore, it should be understood that the total particle count, as well as particle counts in critical PM10 and PM2.5 size categories, are important to monitor as well as to report the range in particle numbers to which patients are exposed. Consequently, tight control of daily particle count exposures is important and mandatory to ensure patient safety and limit the possibility for adverse reactions, particularly in the asthmatic patient.

In the Horak study, the ELISA method was used every hour to measure allergen content. However, once again allergen concentrations over the course of the experiment were not provided and no indication of variation in the allergen content measured indicated. Later studies increased the level and duration of dust mite allergen exposure to 70 ng/m³ over 8 h and 110 ng/m³ over 10 hours total exposure and 5 hours, but as for the initial study no indication of the variation in allergen concentration or particle sizes or counts was provided. Furthermore, although it was stated that the allergen batches were consistent, the particle counts stated varied unpredictably with the allergen content; such that, in the first study 40 ng/m³ was said to be provided on 4000 particles/m³ while later studies stated that a higher allergen load, 70 ng/m³, was provided on fewer particles, 2500 particles/m³. A more recent study did not provide any indication of the target allergen load but stated that it maintained the particle counts at the lowest particle count to date of 1000 particles/m³. No indication of the airborne dust mite allergen concentration or the method of particle counting performed was provided.

In 1997, Ronborg et al. published a study in which asthmatics were exposed to dust mite allergen in a chamber. In this study, a very small, cost-effective, portable chamber was used which had been developed for allergen challenge. This chamber holds only one subject at a time. The method of aerosolization of dust mite allergen involved solubilizing the allergen in buffered saline and human serum albumin (0.3%) and subsequent nebulization of this test solution. This resulted in the total exposure to the allergen over the course of the study to be 1200 ng (if averaged over the course of the entire study, the average exposure is estimated to be 50 ng/m3, however notably exposure would not be constant since there are no mechanisms to maintain this airborne concentration). This chamber's design and mode of exposure results in a profile of dust mite allergen exposure such that the allergen load is not constant over the duration of the study and there is an initial peak of nebulized allergen, followed by a clearing period of several hours. Therefore, the fact that allergen load was not constant over the course of the studies may in part result in some of the patient inconsistencies observed in this study.

In sum, the dust mite allergen studies discussed above, though pioneering, do not provide the level of control of dust mite allergen exposures that are required, even for the study of patients with mild asthma. A dust mite allergen challenge model for the study of asthma will require that the airborne allergen level be tightly controlled and monitored. The duration of the exposure should be maximized within the bounds of tolerability and safety to allow the study of the efficacy of drug therapies but also to provide insights into the nature of the correlation between dust mite allergen load and the symptoms and etiology of asthma.

On the basis of the foregoing, there is a need for a novel method and apparatus for applying dust mite allergen in a controlled manner to elucidate the etiological links between allergen particles and asthma.

### SUMMARY OF THE INVENTION

The present invention provides a method, materials and apparatus for investigating asthma in humans using dust mite allergen.

It is known that dust mite powders need to be of a particular size in order to penetrate the human respiratory tree, namely approximately 10 microns or less in diameter. However, particles that are too small, i.e. less than 2.5 microns in diameter, have a tendency to collect in the lungs. Further, in general, the larger the dust mite particle, the greater the surface area and therefore the greater the allergen content. Finally, it should be borne in mind that it is difficult to effectively aerosolize larger particles.

According to one aspect of the present invention, dust mite culture is used to prepare a dust mite preparation comprising powders of a controlled particle size, the controlled particles size striking a balance between the considerations listed above. In other words, the controlled particle size allows the particles to be respirable in humans, effectively aerosolized and deliver a significant amount of allergen. The present invention optimizes these factors in order to implement dust mite allergen for an asthma challenge model.

According to another aspect of the present invention, dust mite powder of a controlled size is aerosolized into an environmental exposure chamber to elucidate the etiological links between dust mite allergen concentration and asthma response in humans. The environmental exposure chamber is specially-designed to promote the homogenous distribution of the allergen.

Preferably, the dust mite allergen preparation comprises particles having an average diameter of less than 25 microns, and more preferably an average particle size of 5-10 microns. Advantageously, investigation into aerosolized particle number and dust mite allergen concentration has revealed that a very strong correlation exists for particles within the 5-10 micron diameter range. In accordance with yet another aspect of the present invention, this correlation allows for particle counting methods to estimate the allergen concentrations, enabling “real-time” prediction of concentration levels without the use of expensive and time-consuming assay analysis techniques.

### BRIEF DESCRIPTION OF THE DRAWINGS

A detailed description of the preferred embodiments are provided herein below by way of example only and with reference to the following drawings, in which:

FIG. 1 is flowchart illustrating general method steps in accordance with one aspect of the present invention.

FIG. 2 is a flowchart illustrating the steps in correlating allergen measurements with measurements of particle numbers and sizes to yield an estimate airborne allergen concentration.

FIG. 3 is a flowchart illustrating an approach for aerosolization within an environmental exposure chamber to achieve a particular airborne allergen concentration.

FIG. 4 is a graph illustrating sample particle counts obtained in an EEC for different milled dust mite culture particle sizes.

FIGS. 5A and 5B are graphs illustrating sample particle counts obtained in an EEC for 5 and 10 μm particle sizes, respectively.

FIG. 6 is a three-dimensional graph illustrating a volumetric model of allergen concentration in an EEC.

FIG. 7 is a graph illustrating a strong covariation between aerosolized particle number and dust mite allergen concentration for both 5 μm and 10 μm particle sizes.

FIG. 8 illustrates the covariation between particle number and allergen concentration for 0.5 μm particles.

FIG. 9 illustrates the covariation between particle number and allergen concentration for 1 μm and 2 μm particles.

FIG. 10 illustrates the covariation between particle number and allergen concentration for 25 μm particles.

FIG. 11 illustrates the covariation between particle number and allergen concentration for 5 and 10 μm particles.

In the figures, embodiments of the invention are illustrated by way of example. It is expressly understood that the description and drawings are only for the purpose of illustration and as an aid to understanding, and are not intended as a definition of the limits of the invention.

### DETAILED DESCRIPTION OF THE INVENTION

Dust mite allergen for Der p1 is present on spent dust mite cultures from dust mite Dermatophagoides pteronyssinus. The particle size on which the allergen is borne is a critical factor in determining the level to which dust mite allergen can penetrate the human respiratory tree. Dust mite powders from spent dust mite cultures have a preponderance of larger particle sizes greater than 25 microns which are relevant to test respiratory disease of the upper respiratory tract.

However, in an aspect of the present invention, it has been discovered that there is a clear correlation between airborne allergen concentration in a chamber and particle number of dust mite allergen particles sized 5 or 10 microns. The physical basis for this finding is that larger particles have larger surface area such that as the radius of the particle increases by a unit amount, the surface area increases in a squared unit manner. Since the largest particles aerosolized in an EEC, i.e. 25 microns or greater, do not remain airborne appreciably, the next largest particles, 5 and 10 microns (which are stably airborne), carry a significant amount of allergen and their particle number can be used to predict allergen concentration.

In this regard, the controlled particle size is essentially a balance of considerations such that the optimal particles are both of a respirable size and of a size that allows effective aerosolization. This is important and advantageous since this indicates that rather than conducting many costly ELISA analyses, initial and fewer ELISA analyses can be used to “calibrate”the allergen content within an EEC. Also, since ELISA analyses take at least 6 hours to perform, laser counting of critical particle sizes can be used as a real-time indication and approximation of airborne allergen concentration.

The finding that particles sized 5 and 10 microns correlate closely with airborne allergen concentration is particularly advantageous in the EEC setting since these particles can be suspended in a routine and controllable fashion whereas the smallest particles (2.5 microns or less) are easily re-suspended with any movement in the chamber and less controllable. The advantages and appropriateness of the study of dust mite allergen on particles sized 5 to 10 microns are therefore obvious and desirable.

As discussed above, house dust is a strongly allergenic material because it is usually heavily contaminated with the faecal pellets and skins of the dust mite Dermatophagoides. Generally, in order to produce the required materials in accordance with the present invention, spent dust mite cultures are derived from dust mites housed in an open glass box and bred on a diet of baker's yeast set. Generally, faecal pellets and mite skins are harvested and crudely sieved to produce dust mite powders for research purposes. For example, dust mite powder derived from spent dust mite cultures can be purchased from INDOOR Biotechnologies Inc. (U.S.A.). Preferably these powders are desiccated to inhibit particle clumping and decrease average particle size, however some clumping and particle size increases may still occur. As stated, the allergenic component is believed to be proteins of digestion present in mite cultures which have proteolytic activity, specifically Der p and Der f proteins.

Because of the clear importance of particle size with respect to studying asthmatic response, dust mite allergen can be prepared having a controlled particle size. This novel concept comprises preparing the dust mite allergen so that the particles have an average diameter in the respirable range, as discussed, namely less than 25 microns, and preferably 2.5 to 15 microns, and more preferably 5 to 10 microns. In this sense, the dust mite allergen particles are engineered or conditioned to improve the suitability for etiological testing. Without this step, the particle sizes for the dust mite are invariably too large, preventing effective aerosolization from being achieved, and consequently hindering (and perhaps even preventing) the use of airborne dust mite allergen for asthma studies.

In one embodiment of the present invention, mechanical milling is used to condition dust mite allergen powders in order to reduce both particle size and size variation for their use as an experimental inhalant.

Milling is an example of a mechanical means for preparing dust mite allergen particles of a controlled size. In general, milling is a process involving mechanical impaction using hard materials to create fine powders. Ball milling is a common type of milling, involving colliding hard balls with the relevant material, thereby crushing the material to a power. The longer a material is ball milled, the smaller the average diameter of the particles.

However, it should be expressly understood that milling or other mechanical methods are only one means of preparing dust mite powder having a controlled particle size, and the present invention contemplates any other means for either achieving the desired result, namely preparing dust mite allergen particles with an average size of 25 microns or less, or more preferably having an average particle size of 5-10 microns. In this regard, any means of sorting particles is acceptable. For example, a cyclone can be used to “sort” dust mite allergen particles, in a manner that is known. Other sorting methods are known, including for example sieving or use of an elutriator.

Since spent dust mite cultures are hygroscopic, when exposed to air they can become sticky which can lead to reduced product quality and shape and size non-uniformity. Therefore, milling of dust mite cultures is preferably performed under conditions that limits exposure of the milled particles to air, such as an inert environment under low humidity, or in a gaseous environment like nitrogen gas.

Optical particle counting and impactor train sampling can be used to characterize both the initial parent and ball-milled dust mite sample particle size and particle morphology. Size separated samples can be utilized to determine material specific gravity from settling experiments. Quartz particles of known particle size distribution can be used as “standard” particles for phenomenological comparison.

In one particular experiment, the data provided after milling demonstrated that 95% (by particle count) of the parent mite culture was comprised of particles greater than 10 μm with most particles being approximately 30 μm in diameter. Particles with 30 μm diameters are calculated to have a settling time of approximately 30 s for an 8 foot fall and 10-30 s breathing zone residence time. For reasons discussed above, these properties are appropriate for use as an experimental inhalant. The controlled milling of parent material resulted in 98% (by particle count) of the particles being less than 10 μm with irregular particle shapes and with suspension times of minutes to hours. Milled dust mite particles had similar aerodynamic characteristics to standard quartz particles of the same size. In this way, dust mite allergen carrier particles were prepared with known particle size and aerodynamics, such that disease relevant particles sized 5-10 μm are increased and particle size variation is decreased.

According to another aspect of the present invention, the dust mite allergen preparation can then be used within an environmental exposure chamber for the study of dust mite allergen effects on patients. Aerosolization of fine particulate matter is well known in the art. Generally speaking, “aerosolization” refers to any method of converting a powder to a spray or suspension in air. In this regard, the present invention is not limited to any one aerosolization method but contemplates any means to aerosolize the particles into the air of the chamber. What is important is that the aerosolization of dust mite allergen achieves exposure ranges within the EEC that are comparable to that reported for household exposures.

Preferably, the chamber and aerosolization designs allow for the levels of ambient dust mite allergen to remain stable at a specific level. For example, 100 ng/m3 corresponds with levels that have been reported in household bedroom and living areas. Therefore, the use of this chamber to develop a model for asthma testing will provide etiological insights into asthma as well as a therapeutic test model.

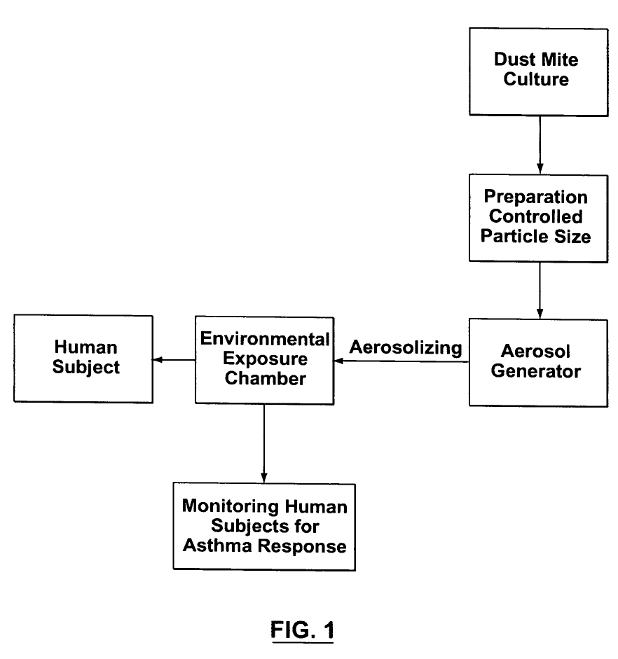

The general method steps for this approach are illustrated in FIG. 1. The development of a model to test therapeutic effectiveness under more natural exposure conditions will provide a significant advance both scientifically and from a drug testing and therapeutic and regulatory stand point.

For the design of an appropriate EEC, regard must be had to certain specifications, namely humidity control, temperature control and HVAC, as well as (i) control of dust mite allergen delivery on respirable particle size, (ii) dust mite allergen aerosolization, (iii) quantification and verification of accuracy dust mite allergen concentration, (iv) quantification of respirable particle size and number, and (v) quantification and correlation analysis between particle size and number and dust mite allergen concentration.

In accordance with one particular embodiment, an EEC is an enclosed space in which airborne particulates, in this case the dust mite allergen, are kept within strict limits. It is a room designed to full Level II Clean Room specifications. In this example, it is a 125 m² room with a seating capacity of 60 subjects. Humidity and temperature are tightly controlled with thermostatic and hygrometric feedback systems. Heating, ventilation and cooling are adjusted to maintain at least 6.5 air exchanges per hour. The room is supplied with clean fresh air via eight ceiling mounted vents. Both air inlets and outlets are fitted with High Efficiency Particulate Air filters (HEPA). This prevents contaminants from being introduced into the controlled environment from the outside. The room is under a slightly positive pressure relative to adjoining areas to ensure no entry of particulate contaminants from these exterior areas into the chamber. In addition, a small directly connected airlock chamber is attached to the chamber which is enclosed on both sides by doors to minimize any transient changes in environmental factors or airborne particulates upon entry or exit from the chamber.

The walls and ceiling of the chamber are covered with a statically dissipative paint which acts to reduce dust mite powder build up on the walls of the chamber and limit this as a dust powder source or reservoir. The floor of the chamber is covered with smooth, resilient, sheet flooring with few seams. The flooring and ceiling curves upward to meet the walls to form rounded corners and baseboards, such that dust collection is minimized. These floor specializations allow the floor to act as a reservoir for settled dust—particularly the largest dust particles (those larger than 25 μm with approximate 8′ settling times of 20 s). Unlike typical clean room standards, dust mite allergen bearing particles larger than 0.3 μm will accumulate (since HEPA filters on chamber air outlets will permit passage of particles <0.3 mm). However, as described in the experimental protocol below, airborne particle numbers and allergen content will be estimated in real-time and particle aerosolization parameters adjusted accordingly to maintain constant airborne allergen concentrations and particle exposures, which do not exceed daily PM10 or PM2.5 EPA standards. Furthermore, the large particles which will accumulate in the floor reservoir are difficult to re-suspend and therefore will not contribute appreciably to airborne particulate levels.

At least one fan is placed in the chamber to create turbulent airflow to prolong dust aerosolization. The number and placement of fans is based on individual chamber characteristics and are determined from measurement of dust mite allergen and particle size and number at various points in the chamber. The aerosol generator is placed behind a wall in the chamber to prevent subject observation of its operation. Additionally, this wall promotes laminar flow about the aerosol generator.

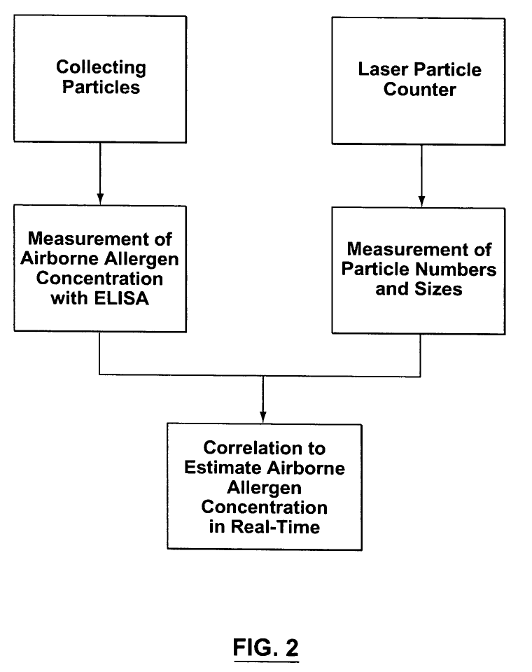

The particle allergen levels can be measured using a volumetric sampler with a glass fiber filter and then using ELISA to quantify the amount of allergen. The particle size and number within the chamber or even within a particular area of the chamber can be quantified using a laser particle counter, for example. Preferably, this can be conducted on a real-time basis. In turn, correlation analysis is carried out to correlate the particle size and number information with the dust mite allergen concentration information obtained using ELISA to yield an estimate of the allergen concentration for the EEC (or an area within the EEC). In this regard, the correlation is essentially used as a calibration means such that the allergen concentration can be estimates for areas of an EEC by collecting particle count information. This can be done substantially in “real-time”, and greatly reduces the number of costly ELISA analyses required. FIG. 2 generally illustrates the steps for this correlation analysis.

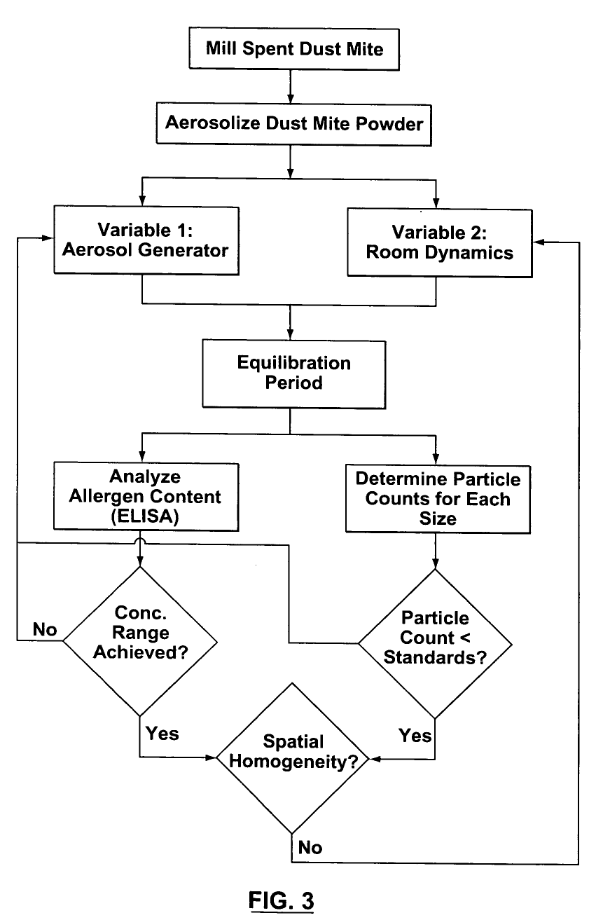

FIG. 3 illustrates a general approach “algorithm” for aerosolizing dust mite powder into an EEC to achieve a desired average dust mite allergen concentration, e.g., 50 to 200 ng/m³, or preferably 80±50 ng/m³. Mill dust mite powder is aerosolized in an EEC. There are two key variables for the control of the dust mite allergen concentration: variable one is the aerosol generation; and variable two is the room dynamics. Variable one depends on, for example, the settings used for the aerosol generator if that is the means used to aerosol the powder into the chamber. Variable two, the room dynamics, depends on, inter alia, the number, speed and placement of fans, the HVAC specifications, and the size and shape of the room.

An equilibration period is required so that the allergen concentration in the chamber environment reaches a steady state, e.g. after 2 or 3 hours. Once this is achieved, the allergen concentration and particle count information can be assessed. If either the concentration range (i.e. less than 80 or greater than 130 ng/m³, for example) or the particle counts are not achieved, then the aerosol generation is adjusted accordingly. If they are within the appropriate range, then spatial uniformity within the EEC is addressed by collecting air samples throughout the chamber. If the allergen concentration within the chamber is not reasonably homogenous, then the room dynamics are altered accordingly. For example, the number of fans and their locations can be altered to obtain spatial uniformity within the chamber.

Particle exposures for PM10 and PM2.5 are generally adhered to such that if particle exposures are too high, the aerosol generator settings are altered. If the dust mite allergen concentration and particle numbers are acceptable, then spatial uniformity within the EEC was addressed by collecting air samples throughout the chamber. Preferably, both the allergen concentration and the particle count analyses are conducted at different locations within the chamber, and move periodically.

As an example, the present invention can be implemented to develop a clinical model to evaluate relative potency of inhaled steroids with mild experimental exacerbation of asthma induced in dust mite allergic asthmatic subjects in an environmental exposure chamber. Preferably, during such a study a chamber in accordance with the present invention maintains the level of dust mite airborne allergen uniformly controlled between 50-120 ng/m3. Such a study would employ an EEC and methodologies required to aerosolize dust mite allergen in a controlled environment such that allergen and particle concentrations are maintained generally homogeneously across the room.

In general, in order to demonstrate equivalence of a second entry inhaled steroid product, it is known that a pharmacokinetic study design is not appropriate, because the drug is locally acting and the systemic levels detected are not necessarily indicative of the topical dose delivered to the pulmonary mucosa. Factors such as particle size and delivery device greatly affect the amount of drug delivered to the lung versus the amount swallowed orally. As a result, a pharmacodynamic response study needs to be used to show therapeutic equivalence. The study design used must be able to reliably show possible differences in relative potency between the test and reference formulations.

It is known that daily low dose allergen challenge in mild to moderate asthmatics induces a measurable reduction in lung function with reduction of FEV1 over a period of 4 to 5 days. Consequently, in accordance with the present invention, an asthma investigation model can be implemented with continuous allergen challenge in an EEC, as opposed to repeated daily low dose allergen challenge.

Such a clinical equivalence study is designed to assess the deterioration of asthma in an EEC asthma model following a short course of oral steroids with two different inhaled doses. This approach has the advantage that the baseline is the inverse of previous study models in that the starting point is best possible control with a short course of oral steroid. Because of this, baseline can be achieved at the end of each period with another course of oral steroid and a crossover study can be done with no detectable carry-over.

This modelling approach comprises a number of advantages, including: (i) controlled allergen exposure results in reduced variability of data produced; (ii) with the lower variability, the sample size will be lower; (iii) the length of the study is generally significantly less than is typical which results in better retention; (iv) observed dosing and lung function measurements results in 100% compliance and very reliable data; and (v) extraneous factors such as seasonal influences and risk of upper respiratory infections which will influence primary endpoint are minimized as the patients are confined.

For example, an EEC is a room (1600 sq. ft.) built to Level II Clean Room specifications where a specially designed dust feeder is used to release spent dust mite feces particles. The air flow and air circulation throughout the room is specially designed to ensure stable levels of ambient dust mite of approximately 100 ng/m³. In milled spent dust mite particles, the majority of Der p1 has been found in particles sized 5 to 10 microns, as discussed above. This size range allowed tight control of allergen aerosolization (little re-suspension), and was in the range of respirable particle size that is important in the etiology of asthma. Preferably, the EEC maintains this level indefinitely.

As described above, the airborne dust mite levels in the EEC can be measured using a volumetric sampler with a glass fiber filter. The levels of allergen are then measured using an ELISA assay to quantify the amount of allergen per meter cube in the air sampled.

The model is developed in two phases, as an example: (i) phase one involves the graduated exposure to dust mite allergen in the EEC with healthy mild asthmatics with increased time over seven stages; and (ii) once safety of exposure to dust mite in the EEC has been established, in phase two 12 subjects will be enrolled in a double blind cross-over study to establish the sensitivity of the model to ascertain the difference between two doses of a standard inhaled steroid.

Phase one will be run in several stages to ensure subject safety. The initial assessment will be limited to a single subject with low level allergen challenge (e.g., 25 ng/m³) of up to 8 hours under careful observation. The time will be increased depending on the tolerance of the subject. Once safety has been established at a low level of allergen exposure, the same procedure will be repeated at moderate level (e.g., 50 ng/m³) of allergen exposure.

For phase two, the study will be conducted as a double blind three way cross-over design with placebo and two doses of oral steroid per day. There will be a qualifying period where subjects will be exposed to house dust mite allergen Der p1 in the EEC for a period of time to ensure that there is deterioration of asthma with exposure (the exact length of exposure will be determined from data obtained from phase one). Following the qualifying period, subjects will be randomized to one of the two steroid doses or placebo. The treatment will be administered once per day in the morning under direct supervision. At the start of each period, subjects will receive a 2 to 3 day course of oral or inhaled steroid at home, to achieve complete control of their asthma. At the end of the course, the subjects will be admitted to the EEC and will be exposed to Der p1 antigen level of approximately 100 ng/m³. Subjects will be continuously in the EEC for a period of 7 days, while being continuously monitored with a physician or paramedic present. While sleeping, subjects will be visually observed for signs of respiratory distress.

Preferably, subjects will be screened according to reasonable inclusion and exclusion criteria.

The EEC will be equipped for overnight stay with beds; washroom and shower facilities will be available just outside the EEC. During the stay in the EEC subjects will have various measurements taken at pre-determined intervals, e.g., spirometry measuring FEV1, FEF 25-75 etc., every hour for the first 6 hours then every four hours while awake.

### EXAMPLE

Cultured spent dust mite cultures were acquired from a supplier (in this case INDOOR Biotechnologies Inc. of Charlottesville, Va., U.S.A.). These cultures were conditioned such that dust mite culture particles are size reduced with the majority of the particles to be less than 25 μm, and generally in the range of 5-15 μm. Particle size reduction was achieved with ballmilling under low humidity conditions (or other inert gas conditions) suitable for hygroscopic powders. Milled dust mite cultures were stored at room temperature and desiccated to prevent clumping.

Milled dust mite cultures were aerosolized with an aerosol generator within an EEC, as described above, with the dust mite particle aerosolization maintained with generation of turbulent flow within the chamber by at least one fan placed strategically in the chamber.

The number of fans and their position within the chamber which are required for maintenance and spatial distribution of dust mite allergen is determined by examining the airborne dust mite allergen and particle numbers, in the manner described above. The airborne dust mite allergen concentration, at least initially, is determined by the setting on the aerosol generator such that the rate of aerosolization must be adjusted to obtain ideal dust mite allergen bearing particle release. Airborne dust mite allergen concentration was determined by collecting air samples during the course of the study using high volume air samplers and analyzing these samples using ELISA. Specifically in this case, the dust mite allergen Der p 1 was measured.

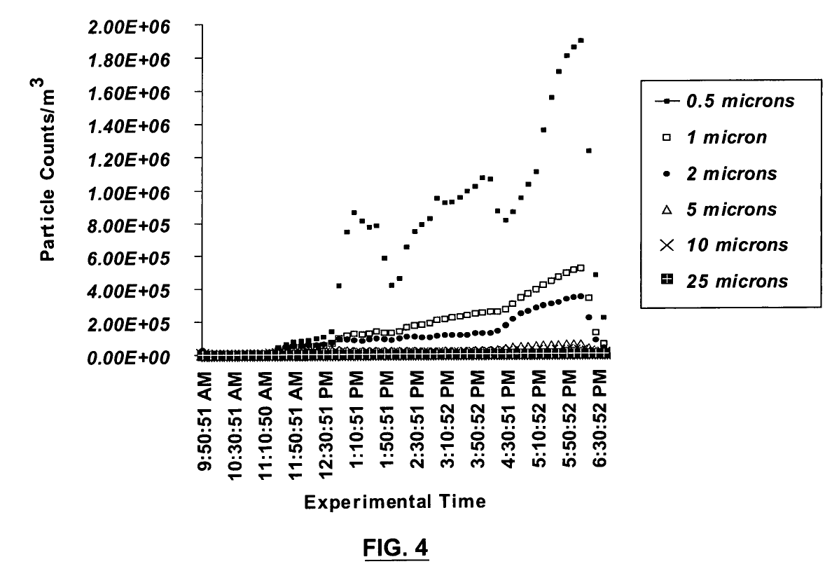

The numbers of airborne particles in each size range of 0.5, 1, 2, 5, 10, and 25 μm was determined using LASAIR II™ particle counter (manufactured by Particle Measuring Systems of Boulder, Colo., U.S.A.). FIG. 4 illustrates the particle counts obtained in the EEC for different milled dust mite particle sizes.

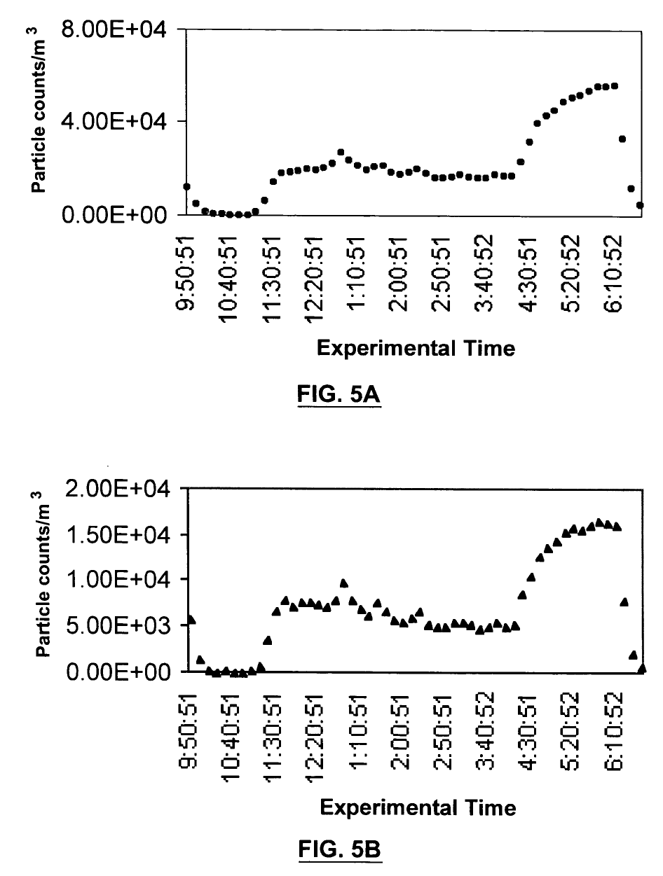

FIG. 5A illustrates the particle count profile for 5.0 μm particles. The particle count initially increased when the aerosol generator was turned on at approximately 11:20. After an equilibration period of 2.5 h, the average particle count could be obtained over 1 hour (2:30-3:30).

FIG. 5B illustrates the particle count profile for 10.0 μm particles. The particle count for 10 μm particles followed the same time profile as for 5 μm particles, however there were approximately 20-fold fewer particles aerosolized for 10 μm particles compared to 5 μm particles.

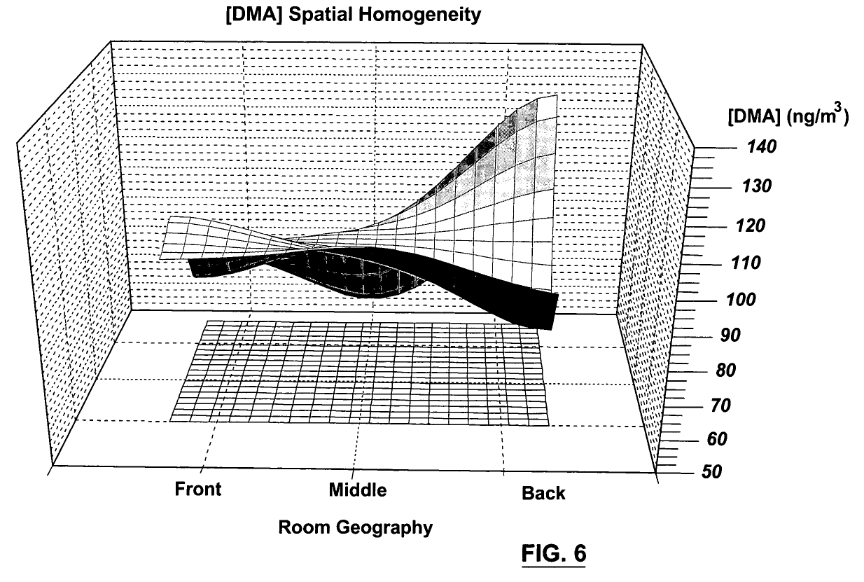

One objective in these studies was to obtain airborne dust mite allergen concentrations of 80±50 ng/m³ and to obtain spatial uniformity for this targeted airborne dust mite allergen at a patient seated eye level, which is generally 48″ above chamber floor. FIG. 6 illustrates that (using the experimental algorithm as discussed with reference to FIG. 3) the spatial uniformity throughout an EEC can be achieved within the etiologically relevant dust mite allergen exposure range. In this case, the mean dust mite allergen concentration (“[DMA]”) was 85.4±5.7 ng/m³ with a maximum value of 115 ng/m³ and a minimum value of 50.7 ng/m³. The allergen concentration values were connected with volumetric smoothing (created using PSI-PLOT™, v.7.01, Poly Software International of Pearl River, N.Y., U.S.A.).

Another objective was that these airborne dust mite allergen concentrations were to be achieved without exceeding the PM10 and PM2.5 EPA particle exposure guidelines. Calculations over the course of a 6-hour exposure demonstrated that levels were well below the recommended levels for PM10 and PM2.5, with 12.30±0.51 ng/m³ and 1.86±0.30 ng/m³ recorded, respectively.

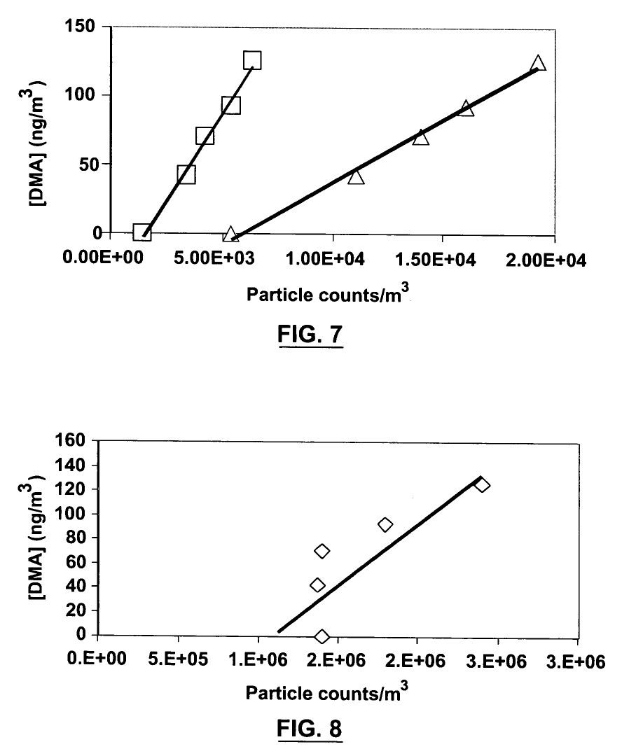

Finally, as this was for the purposes of investigating the asthma response to dust mite allergen, the allergen should be carried primarily on disease-relevant sized particles of 5 to 10 μm. In this case, there was a strong correlative relationship between airborne dust mite allergen concentration and the average particle counts for the 5 and 10 μm sized particles in the dust mite preparation. FIGS. 7 to 10 demonstrate this point graphically.

In particular, FIG. 7 illustrates a strong covariation between aerosolized particle number and dust mite allergen concentration for both 5 μm (indicated with “▴”) and 10 μm particles sizes (indicated with “▪”), with r2=0.99 for both. These correlations were significant (p<0.05). Using this correlation, the airborne dust mite allergen concentration could be accurately estimated from particle counts for 5 and 10 μm size particles.

FIG. 8 illustrates the covariation between particle number and dust mite allergen concentration for 0.5 μm particles revealed r2=0.67, which were not significant.

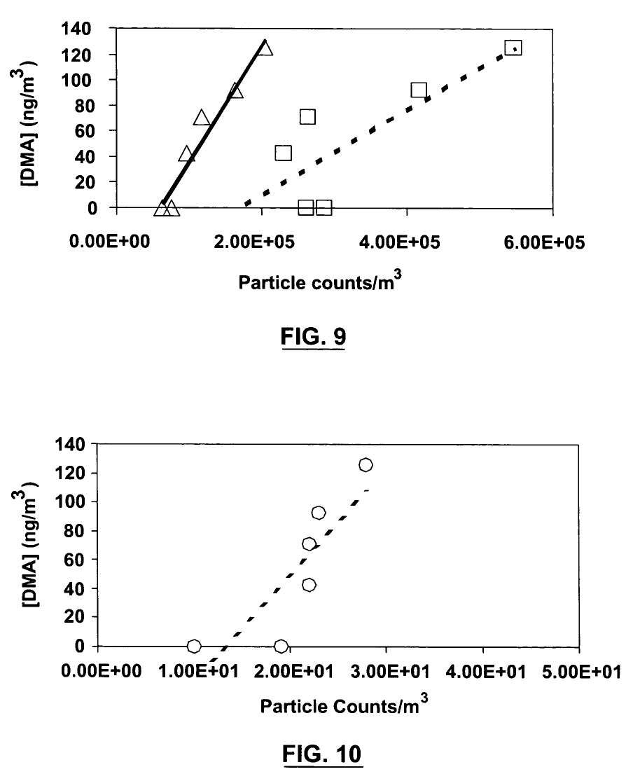

FIG. 9 illustrates the covariation between particle number and dust mite allergen concentration for 1 μm (indicated with “▪”) and 2 μm particles (indicated with “▴”) revealed r2=0.64 and 0.92, respectively, which were both not significant.

Finally, FIG. 10 illustrates the covariation between particle number and dust mite allergen concentration for the largest (25 μm) particles revealed r2=0.67, which was not significant.

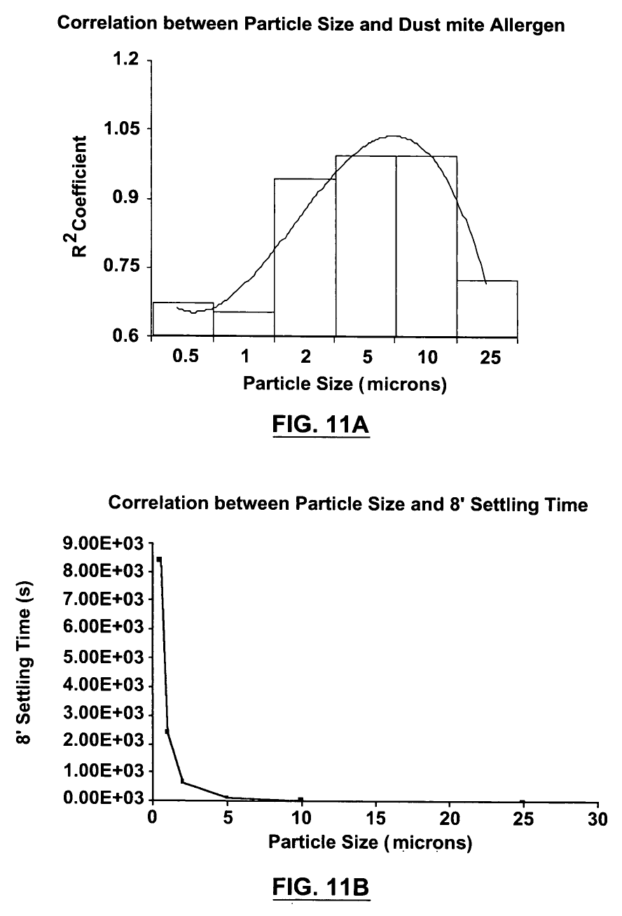

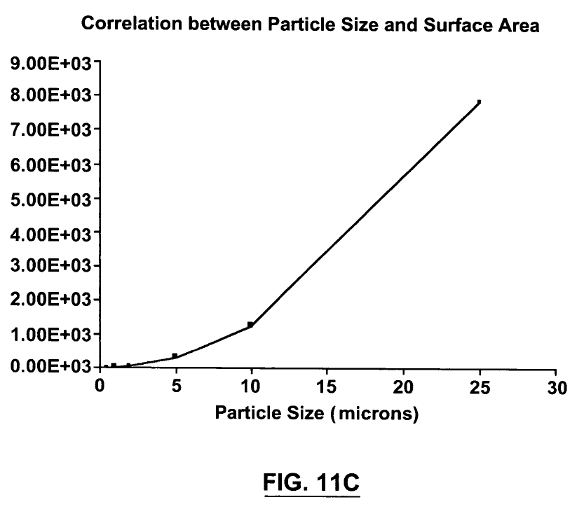

FIG. 11A summarizes the results of FIGS. 7 to 10 by demonstrating that the strongest correlation exists between the 5 and 10 μm sized particles and the dust mite allergen. This correlation is likely at least in part due to the biophysical properties of the milled dust mite particles such that more dust mite allergen is carried on larger particles. In FIG. 11B, the settling time is compared to particle size. In FIG. 11C, particle size is correlated with surface area (given in μm2). Clearly, the optimization of all FIGS. 11A, 11B and 11C occur for particles having a diameter of 5 to 10 microns.

Retrospective analyses of experiments demonstrate that the dust mite allergen concentration, measured by ELISA, was not significantly different from those estimated using 5 and 10 μm particle count calibrations, and hence in this well-defined system using milled dust mite cultures the airborne dust mite allergen concentration could be accurately estimated from particle numbers specifically for disease-relevant particles sized 5 and 10 μm, being 82.7±11.5 ng/m³ and 75.0±20.4 ng/m³, respectively.

These studies indicate that these methods can be used to achieve a well controlled level of airborne dust mite allergen exposure in a natural setting that is more representative of typical dust mite allergen exposures in the household and elsewhere. These methods can be used to estimate and monitor dust mite allergen exposure in substantially real-time, on a minute-byminute basis. This is important for the development of an EEC model in which long-standing, low level exposures will be required to study asthma in a safe and well-tolerated manner.

What is claimed is:

1. A method for investigating asthma in a human being, comprising the steps of:
(a) placing the human being into an environmentally controlled chamber;
(b) preparing a dust mite allergen preparation, the dust mite allergen preparation consisting of particles having an average particle diameter of 1 to 25 microns;
(c) introducing a predetermined amount of the dust mite allergen preparation into the environmentally controlled chamber, thereby exposing the human being to a controlled level of the dust mite allergen preparation; and
(d) monitoring the human being for an allergenic response to the dust mite allergen preparation.

2. The method of claim 1, wherein the dust mite allergen preparation consists of particles having an average particle diameter of 5 to 10 microns.

3. The method of claim 1, wherein the dust mite allergen preparation is prepared by mechanical milling.

4. The method of claim 1, wherein the dust mite allergen preparation is introduced into the environmentally controlled chamber by an aerosolizing means.

5. The method of claim 1, wherein the dust mite allergen preparation is dispersed in a controlled manner substantially uniformly throughout the environmentally controlled chamber.

6. The method of claim 5, wherein the dust mite allergen preparation has a concentration in the environmentally controlled chamber in the range of 50 ng/m³ to 200 ng/m³.

7. The method of claim 1, comprising the further steps of:
(a) determining an airborne allergen concentration within the environmentally controlled chamber; and
(b) correlating the airborne allergen concentration with the allergenic response.

8. The method of claim 1, further comprising:
(a) collecting an air sample in the environmentally controlled chamber, the air sample comprising particles of the dust mite allergen preparation;
(b) measuring the air sample to determine an allergen concentration using an enzyme-linked immuno-sorbent assay method;
(c) using a particle counter to gather particle information in the environmentally controlled chamber; and
(d) correlating the allergen concentration and the particle information to determine a calibration relationship.

9. The method of claim 8, further comprising:
(a) using the particle counter to gather additional particle information; and
(b) using the calibration relationship and the additional particle information to determine an estimated chamber allergen concentration.

10. The method of claim 9, wherein the estimated allergen concentration is calculated on a substantially real-time basis.

11. The method of claim 9, wherein the particle information is gathered in two or more locations in the environmentally controlled chamber.

12. The method of claim 1, wherein the environmentally controlled chamber comprises at least one fan to provide generally turbulent flow within the environmentally controlled chamber, a temperature control means, a ventilation means, an internal wall to promote laminar airflow within the environmentally controlled chamber, statically dissipative wall surfacing, and smooth and rounded corners between walls and ceiling of the environmentally controlled chamber.

13. A method for investigating asthma in a human being, by exposing the human being to a dust mite allergen preparation and monitoring an allergic response thereto in the human being, wherein the dust mite allergen preparation comprises particles prepared by mechanical fabrication, said particles having an average diameter in the range of 2.5 to 15 microns.

14. The method of claim 12, wherein the mechanical fabrication is milling.

15. The method of claim 12, wherein the dust mite allergen preparation is derived from Dermatophagoides pteronyssinus or Dermatophagoides farinae, or a combination thereof.

16. A method for investigating asthma in a human being by exposing the human being to a dust mite allergen preparation and monitoring an allergenic response thereto in the human being, wherein the dust mite allergen preparation comprises particles prepared and sorted into an average particle diameter range of between 2.5 to 15 microns.

17. The method of claim 15, wherein the dust mite allergen preparation is derived from Dermatophagoides pteronyssinus or Dermatophagoides farinae, or a combination thereof.

18. A chamber for investigating asthma in a human being, comprising:
(a) at least one fan for moving air;
(b) a heating and cooling means to maintain the chamber at a constant temperature;
(c) a ventilation means for substantially reducing particulate matter content in air entering the chamber; and
(d) an aerosolizing means for dispersing a dust mite allergen preparation, the dust mite allergen comprising particles having an average diameter in the range of 2.5 to 15 microns;
wherein the chamber is operable to observe an allergenic response in the human being.

19. The chamber of claim 18, wherein the particles of the dust mite allergen preparation are analyzed with a particle counter to determine particle size and number.

20. The chamber of claim 18, wherein the aerosolizing means comprises a fluid bed aerosol generator.

21. The chamber of claim 18, having an internal wall to promote laminar airflow within the chamber and statically dissipative wall surfacing.

22. The chamber of claim 18, having smooth and rounded room corners between walls and ceiling.
